(19) United States
(12) Patent Application Publication
(10) Pub. No.: US 2007/0276350 A1
(43) Pub. Date: Nov. 29, 2007

(54) Absorbent Article

(76) Inventors: Young-Man Park, Seoul (KR); Seong-Choon Lee, Seoul (KR); Yong-Joo Tcha, Kyungki-do (KR)

Correspondence Address: Ladas & Parry, 5670 Wilshire Boulevard Suite 2100, Los Angeles, CA, 90036-5679, US

(21) Appl. No.: 10/562,588

(22) PCT Filed: Jun. 24, 2004
(86) PCT No.: PCT/JP04/09292

§ 371 Date: May 30, 2006

(30) Foreign Application Priority Data
Jun. 27, 2003 (JP) 2003-185447

(51) Int. Cl. A61F 13/84

(52) U.S. Cl. 604/385.13

(57) ABSTRACT

An absorbent article having a fastening sticker which cannot be drawn out by its wearer, such as an infant wearing it, but is freely attachable to and detachable from the outer surface of the main body of the article, can be used whenever and as often as necessary, and has a very wide range of uses including the tightening of the paper diaper on the body of its wearer, the repair of its side when its side seal is broken, and moreover the intellectual training of the infant, and an absorbent article having a front body side and a back body side formed by folding at the longitudinal middle point of the main body of the article having an absorbent inserted between a liquid-permeable top sheet on the body side and a back sheet on the outside, the absorbent article having a fastening sticker to be used for its disposal, characterized in that the fastening sticker is formed from a disposable sheet attached to the outer surface of the article, such as a sheet-like member capable of being re-stuck to and re-peeled off the outer surface of the back sheet and separable completely from the main body of the article.

### TECHNICAL FIELD

This invention relates to an absorbent article, and more particularly to an absorbent article having a fastening sticker which can be used for many purposes including not only the disposal of the article, but also the tightening or repair of the article or the intellectual training of an infant.

### BACKGROUND ART

Various things having different shapes depending on their purposes or uses have been generally known and put to practical use for absorbent articles, such as disposable paper diapers, sanitary napkins or various kinds of pads, but it can be said that the main bodies of those articles do not greatly differ from one another in basic construction.

For example, an absorbent article as shown by a top plan view in FIG. 1, i.e. a pants type disposable paper diaper 1 is a pants type paper diaper made as shown in a perspective view of FIG. 2 by arranging on a back sheet 2 formed from polyethylene, etc. a plurality of elastic stretchable members 3 and 4 formed from rubber strings, etc. along its front and back waist portions, a first elastic stretchable member 6 extending from the edge of the front body side F around one leg to the edge of the front body side F around another leg through the bottom portion of the main body 5 of the diaper and a second elastic stretchable member 7 extending from the edge of the back body side B around one leg to the edge of the back body side B around another leg through the crotch portion of the main body 5 of the diaper, placing an absorbent 8 on the back sheet 2, superposing thereon a liquid-permeable top sheet 9 formed from a non-woven fabric, etc., folding the main body 5 of the diaper shaped like an hourglass at its centerline C-C at around the longitudinal central portion as shown by an arrow Y0 and joining its side edge portions together by heat seal or ultrasonic welding, etc.

Known as other modified examples are one having a three-dimensional or plane gathers formed on the main body 5 of the diaper and one having a water-proofing film formed on the inside of the back sheet 2, depending on its purpose or use, etc.

The disposable paper diaper 1 has a member used for its disposal, or a fastening tape 10 for disposal, protruding from the surface of the back sheet 2 situated on the upper part of the center of the back body side B, as shown in FIG. 2. The fastening tape 10 for disposal is usually fixed by heat seal, etc. at one end 10a to the upper surface of the back sheet 2, folded on itself plural times in its middle portion 10b, and stuck with an adhesive 11 in a separable way, as shown in FIG. 3, a sectional view taken along the line X-X of FIG. 2, so that it can be gripped at its distal end 10c and extended. The fastening tape 10 for disposal is used in such a way that when the diaper 1 is going to be disposed, it may be picked up at its distal end 10c, extended in the direction of an arrow Y1, wound about the diaper 1 to make it compact, and fastened to its back end (see, for example, Official Gazette JP-UM-A-6-77722).

It has, however, been a case with the disposable paper diaper 1 having the fastening tape 10 for disposal as described above that the tape 10 protruding from the outer surface of the back sheet 2 is touched by the hand of its wearer, particularly an infant wearing it and pulled unintentionally. Once that the fastening tape 10 for disposal is extended, it has been a case that the fastening tape 10 is suspended from the paper diaper 1, so that its sticky portion 11 adheres here and there. It results not only making the disposal of the diaper impossible, but also it looks unpleasant. Further, the fastening tape 10 for disposal is intended only for the use of the disposal of the diaper, and has not had another special purpose.

This invention has been made under the circumstances as described above, and its object is to provide an absorbent article having a fastening sticker which cannot be drawn out by its wearer, such as an infant wearing it, but is freely attachable to and detachable from the outer surface of the main body of the article, can be used whenever and as often as necessary, and has a very wide range of use including the fastening of the paper diaper on the body of its wearer, the repair of its side when the side seal is broken, and moreover the intellectual training of an infant.

### DISCLOSURE OF THE INVENTION

The above object of this invention is attained by providing an absorbent article having a front body side and a back body side formed by folding at the longitudinal middle point of the main body of the article having an absorbent inserted between a liquid-permeable top sheet on the body side and a back sheet on the outside, the absorbent article having a fastening sticker to be used for its disposal, etc., characterized in that the fastening sticker is formed from a disposable sheet attached to the outer surface of the article, such as a sheet-like member capable of being re-stuck to and re-peeled off from the outer surface of the back sheet and separable completely from the main body of the article.

The above object of this invention is effectively attained by providing an absorbent article characterized in that the fastening sticker is fastened to the front or back body side.

The above object of this invention is effectively attained by providing an absorbent article characterized in that the fastening sticker has a design on its surface.

The above object of this invention is effectively attained by providing an absorbent article characterized in that the design is aiming the intellectual education of an infant.

The above object of this invention is effectively attained by providing an absorbent article characterized in that the design of the fastening sticker is relating to the pattern of a design on the outer surface of the main body of the article.

The above object of this invention is effectively attained by providing an absorbent article characterized in that a part of the fastening sticker on which the design is drawn or any other portion thereof is separable from the base portion of the sticker along perforations.

The above object of this invention is effectively attained by providing an absorbent article characterized in that the fastening sticker has a stretchable sheet material in its middle portion and that the sheet-like member capable of being re-stuck and re-peeled off and separable completely from the main body of the article is connected to opposite end portions of the stretchable sheet material.

The above object of this invention is effectively attained by providing an absorbent article characterized by having a plurality of such fastening stickers.

Moreover, the above object of this invention is effectively attained by providing an absorbent article characterized in that the fastening sticker is attached to or enclosed in a product package for the absorbent article.

According to the absorbent article of this invention (hereinafter called the “present absorbent article”), the fastening sticker fastened removably to the outer surface of the back sheet (particularly the outer surface of the front or back body side of the back sheet) can be peeled off for use whenever and as often as the after-treatment, putting in order of the present absorbent article or the repair of a broken part thereof is, for example, necessary. As the fastening sticker is formed from a flat sheet material and does not have any catching portion, there is no fear of the wearer, such as an infant, having his hand caught thereby and extending it out.

Referring particularly to the present absorbent article having a design (particularly a design composed of a pattern aimed at the intellectual education of an infant) drawn on the surface of the fastening sticker, it not only has the effects as stated above, but also makes an infant, etc. willing to wear it and makes the change of diapers enjoyable under the intellectual education.

The present absorbent article in which the design is a design related in pattern to a design drawn on the outer surface of the article main body provides an excellently designed paper diaper not giving any sense of incongruity and makes it possible to enhance the interest of an infant in the design to a further extent.

The present absorbent article in which that portion of the fastening sticker on which the design is drawn, or any other portion thereof is separable from the base of the sticker along perforations enables a separated sheet to be used for the intellectual education of an infant or as a toy to be played with.

Referring particularly to the present absorbent article in which the fastening sticker has a stretchable sheet material in its middle portion, the stretchable sheet material can be used for fastening the present absorbent article still more strongly.

The present absorbent article having a plurality of such fastening stickers on the article main body provides a greater number of chances for use as the after-treatment, putting in order or the repair of the article and thereby enables its disposal in a compact shape.

Moreover, the supply of the fastening sticker in a product package for the present absorbent article improves the convenience for its user.

### BRIEF DESCRIPTION OF THE DRAWINGS

FIG. 1 is an developed top plan view of a known paper diaper.

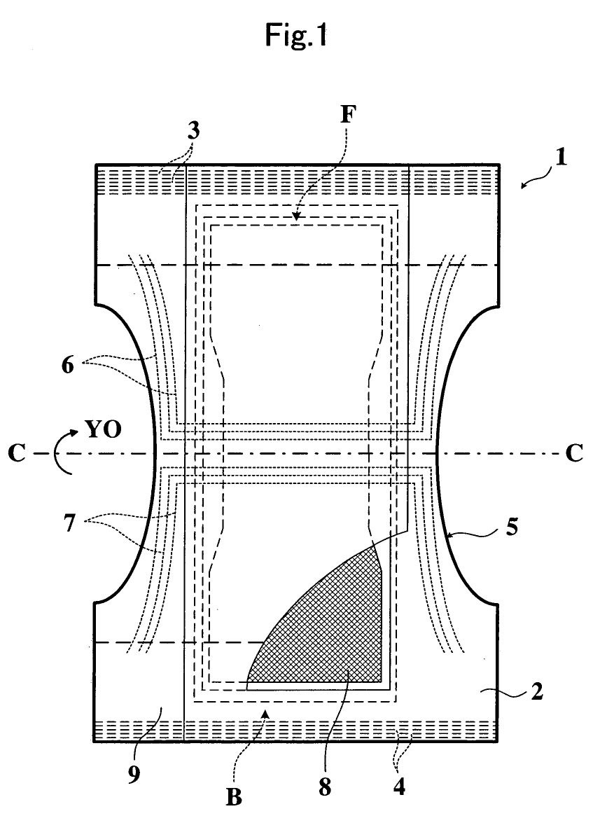

FIG. 2 is a perspective view of an assembled form of the paper diaper shown in FIG. 1.

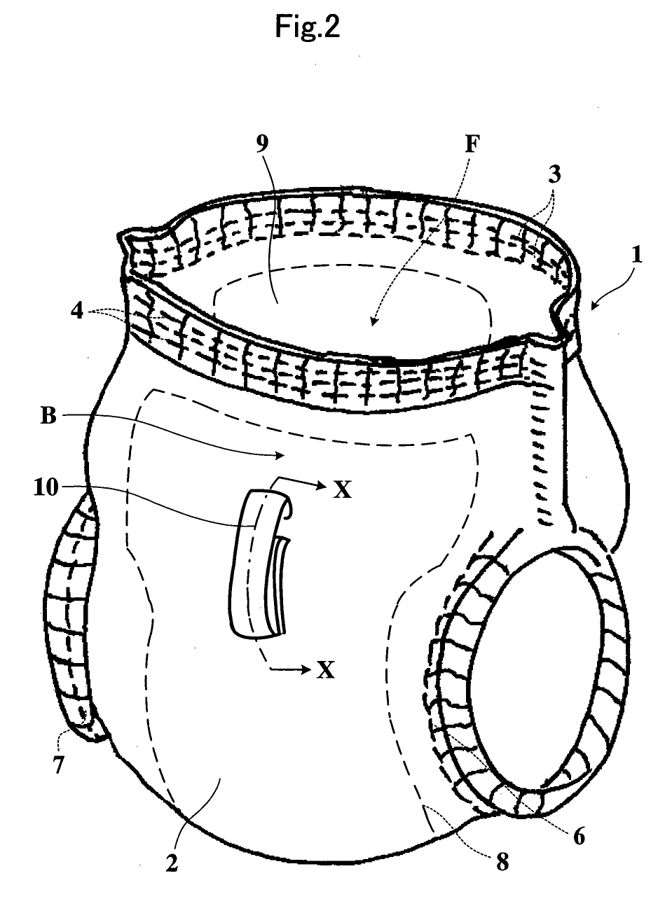

FIG. 3 is a sectional view taken along the line X-X of FIG. 2.

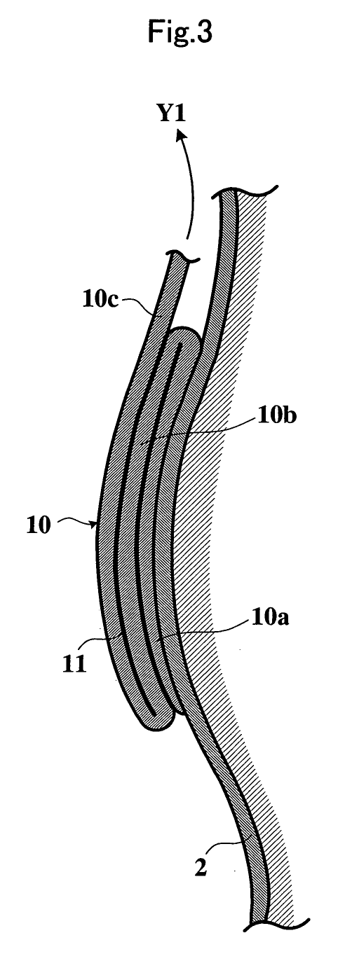

FIG. 4 is a perspective view of a paper diaper embodying this invention.

FIG. 5 is a front view of a flatly folded form of the paper diaper shown in FIG. 4.

FIG. 6 is a perspective view of a fastening sticker forming an essential part of this invention.

FIG. 7 is a cross-sectional view of the fastening sticker shown in FIG. 6.

FIG. 8 is a perspective view of the paper diaper in its shape in which it is ready to be disposed.

FIG. 9 is a perspective view showing a modified form of a fastening sticker.

FIG. 10(A) is a top plan view and FIG. 10(B) is a side view showing a modified form of a fastening sticker.

FIG. 11(A) is a top plan view and FIG. 10(B) is a side view showing a modified form of a fastening sticker.

FIG. 12 is a front view showing a modified form of a fastening sticker.

FIG. 13 is a perspective view showing the state in which fastening stickers are enclosed in a pocket attached to a product package.

### BEST MODE OF CARRYING OUT THE INVENTION

While the details of this invention will now be described by way of an example in which the absorbent article is a disposable paper diaper, it is needless to say that this invention is not necessarily limited to this example, but that variations may be made without departing from the scope of claims.

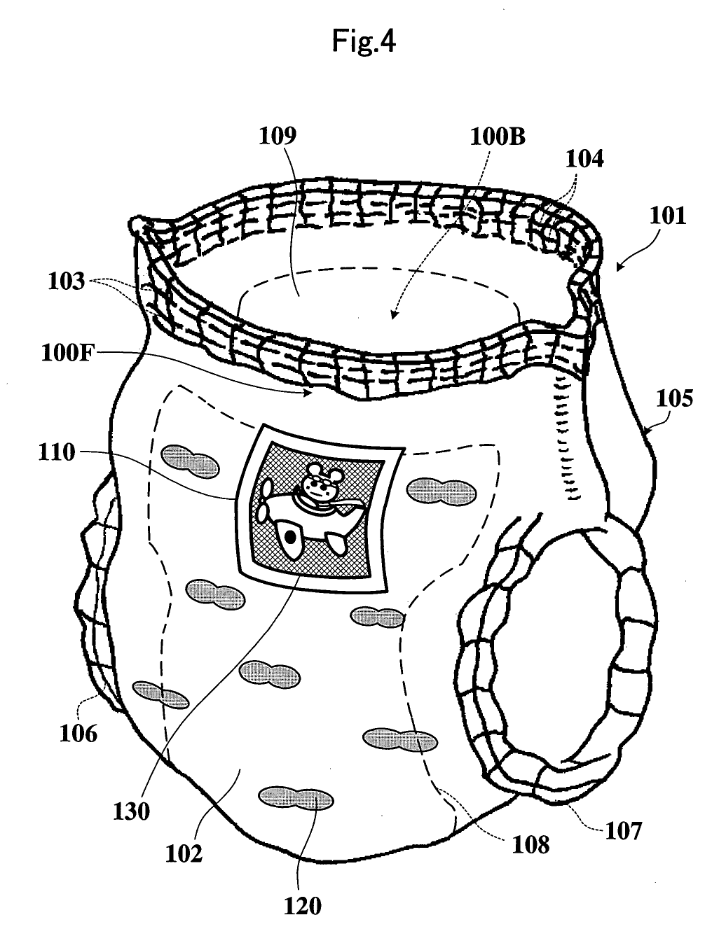

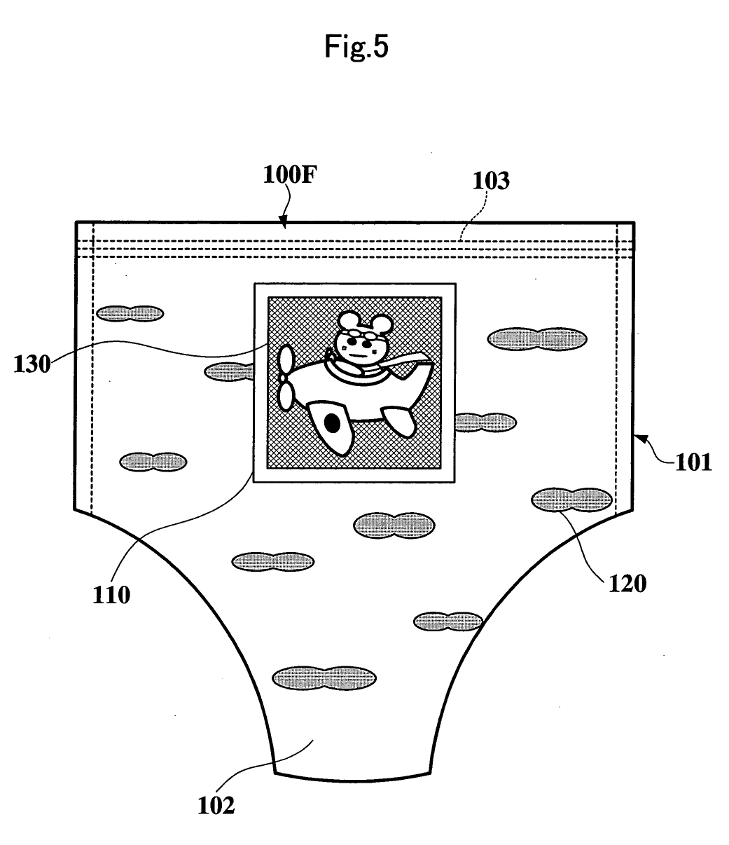

FIG. 4 is a perspective view showing an assembled form of a pants type disposable paper diaper 101 according to one embodiment of this invention (hereinafter referred to as the “present paper diaper”), i.e. its worn form, and FIG. 5 is a view showing the paper diaper 101 folded flatly and viewed from the back body side 100B side. The present paper diaper 101 is substantially identical to the known paper diaper 1 described hereinbefore in any structural aspect other than a part of its structure consisting mainly of a fastening sticker 110 which will be described hereinafter, and its structural parts corresponding to those of the paper diaper 1 will, therefore, be described by employing the same reference numerals plus 100.

The present paper diaper 101 is, like the paper diaper 1 described hereinbefore, a pants type paper diaper made as shown in a perspective view of FIG. 4 by arranging on a back sheet 102 formed from polyethylene, etc. a plurality of elastic stretchable members 103 and 104 formed from rubber strings, etc. along its front and back waist portions, a first elastic stretchable member 106 extending from the edge of the front body side 100F around one leg to the edge of the front body side 100F around another leg through the bottom portion of the main body 105 of the diaper and a second elastic stretchable member 107 extending from the edge of the back body side 100B around one leg to the edge of the back body side 100B around another leg through the crotch portion of the main body 105 of the diaper, placing an absorbent 108 on the impermeable back sheet 102, superposing thereon a liquid-permeable top sheet 109 formed from a non-woven fabric, etc., folding the main body 105 of the diaper shaped like an hourglass back and front at around the longitudinal central portion and joining its side edge portions together by heat seal or ultrasonic welding, etc.

The present paper diaper 101 has designs 120 featured by symbolized cloud-shaped patterns and printed here and there on the outer surface of its main body 105, i.e. on the whole outer surface of the back sheet 102, and a fastening sticker 110 fastened to the outer surface of the back sheet 102 in the upper portion of the central portion of the front body side 100F.

The fastening sticker 110 is formed by a rectangular base sheet 111 having a side length of about 5 cm and formed from a plastic film, and has rectangular plane fasteners 112a and 112b bonded to its back surface along its upper and lower ends, respectively. Thus, the fastening sticker 110 is formed from a disposable sheet having sheet-like members, i.e. the plane fasteners 112a and 112b, capable of being re-stuck to and re-peeled off from the outer surface of the back sheet 102 and separable completely from the article main body 105.

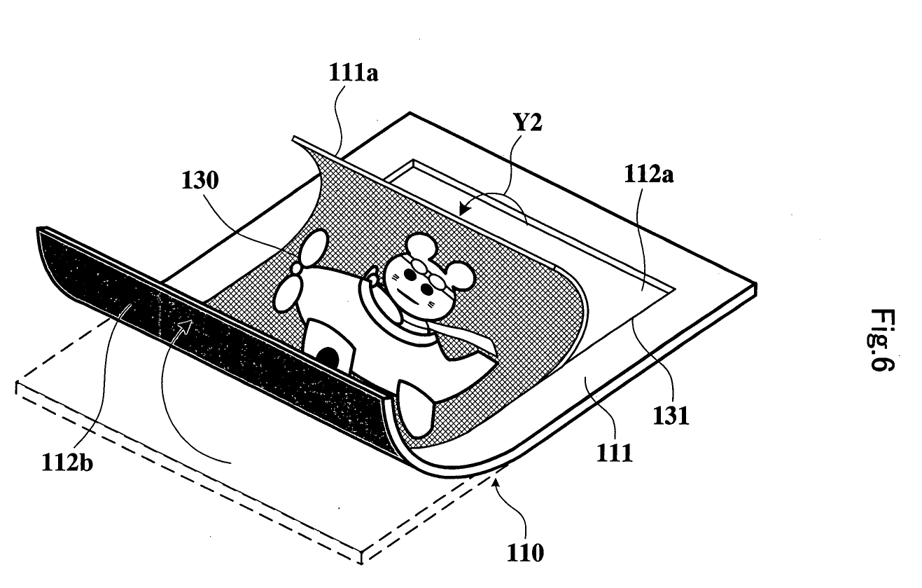

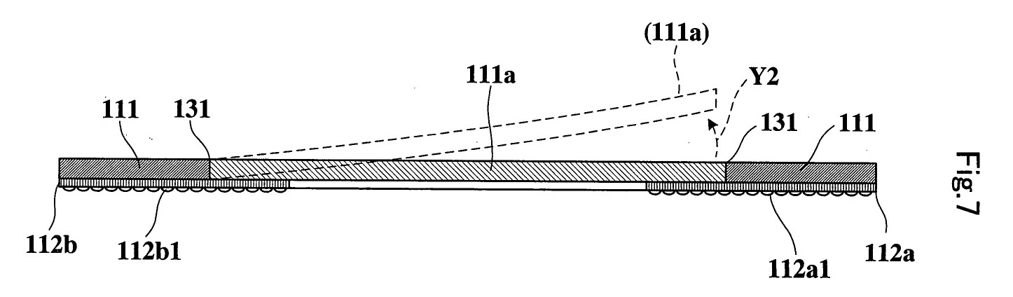

A design 130 symbolizing an animal (little bear) on an airplane is printed in the central portion of the base sheet 111. A sheet 111a having the design 130 drawn thereon has a perforated line 131 made along its outer periphery so as to be capable of being peeled off from the plane fasteners 112a and 112b as shown by an arrow Y2, while leaving the outline portion of the base sheet 111 behind, as shown by a perspective view of FIG. 6 and by a cross-sectional view of FIG. 7. The sheet 111a as peeled off serves as a kind of sticker and is used for the intellectual education of an infant wearing the diaper, or as a toy to be played with.

The designs 120 and 130 can be drawn by any well-known printing method and the designs to be drawn can be selected as desired from any pictures, patterns or characters, depending on the purpose thereof. It is desirable for the interest of any infant and the design of the paper diaper that the designs 120 and 130 be so related to each other as not to give any sense of incongruity.

The plane fasteners 112a and 112b situated apart from each other on the back of the fastening sticker 110 are known ones having hook members having a hook-like or mushroom-shaped cross section on their surfaces, i.e. male members 112a and 112b, respectively. A mechanical seal is formed by the male members 112a and 112b connected with loop-shaped engaging members formed by the back sheet 102, i.e. female members, whereby the fastening sticker 110 is removably fastened to a given location on the outer surface of the back sheet 102. Accordingly, the fastening sticker 110 can be peeled off and used whenever it is required, and as often as it is required.

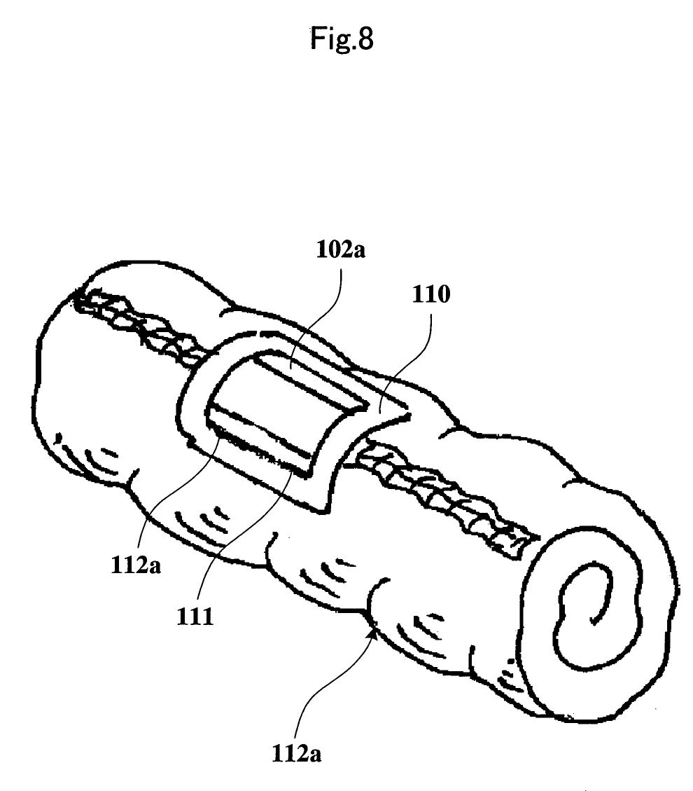

When a person disposes the present paper diaper 101 having its construction as described above, he first peels off the fastening sticker 110 from the given position of the back sheet 102 and also the sheet 111a from the plane fasteners 112a and 112b, and rolls the present paper diaper 101 and fastens its rear end with the fastening sticker 110, as shown by a perspective view of FIG. 8.

The fastening sticker 110 can not only be used for disposing the present paper diaper 101, but can also be used for other purposes, for example, in the case that the present paper diaper 101 is too loose to be worn, its loose portion may be tucked up and fastened with the fastening sticker 110 to avoid the loosening, since the fastening sticker 110 on the present paper diaper 101 is fastened flatly to the outer surface of the paper diaper main body 105, i.e. on the outer surface of the back sheet 102 (without protruding as in any paper diaper hitherto available) and adapted for use whenever and as often as it is required, as described above. It also has the advantage of being usable for the intellectual education of an infant or as a toy to be played with, since the sheet 111a having the design 130 drawn thereon can be used as a kind of sticker preferred by infants, as described above. In the event that its side seal portion has, for example, been broken when the present paper diaper 101 is worn (by a wearer), the fastening sticker 110 can be used to repair the side portion.

Another advantage is that infants who have to wear the diaper, for example, prefer to wear it and the change of diapers becomes enjoyable, since the design 120 on the paper diaper main body 105 and the design 130 on the fastening sticker 110 are related to each other, i.e. the designs represent a picture of an airplane flying in the air in which clouds are floating.

While the details of this invention have been explained based on one embodiment, this invention can be modified in its features as described hereinafter.

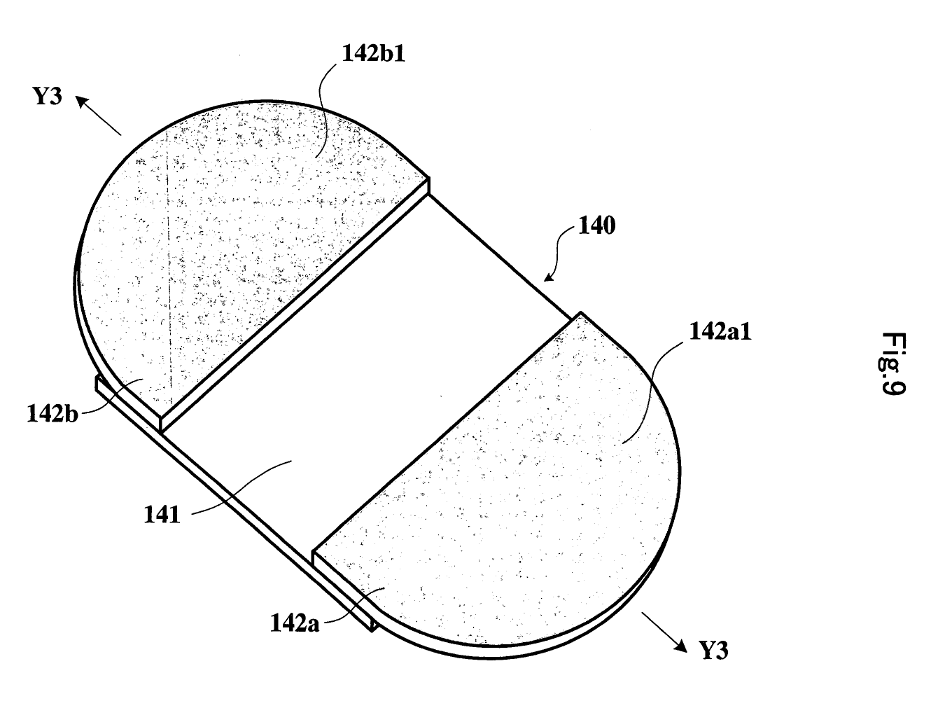

FIG. 9 is a perspective view of a fastening sticker 140 according to a modified form of this invention, as viewed from its back side.

The shape of the fastening sticker 140 is oval, and it has a stretchable rectangular base sheet 141, such as a plastic or rubber sheet, in its middle portion, and semi-circular plane fasteners 142a and 142b having male members 142a and 142b bonded to the base sheet 141 at both ends thereof, respectively, on their back surfaces, as shown in FIG. 9. While the present fastening sticker 140 featured as described above is fastened to the outer surface of the front body side 100F or the back body side 100B of the paper diaper main body 105, i.e. on the outer surface of the back sheet 102, like the former embodiment described hereinbefore, and when it is used, although it is also peeled off from its fastened position like the former embodiment as described hereinbefore, since the present fastening sticker 140 has the stretchable base sheet 141 in the middle portion which allows it to be stretched in opposite directions as shown by arrows Y3, it can be fastened more firmly. Accordingly, the paper diaper 101 as described hereinbefore can be rolled still more compactly for storage or disposal, and can be fastened still more strongly against the loosening when it is worn.

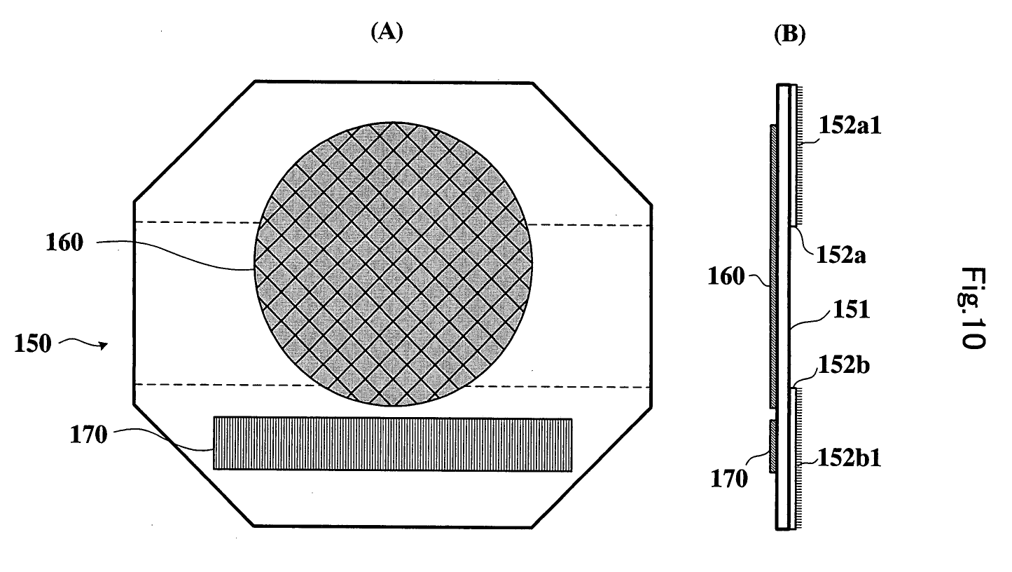

FIG. 10 shows a fastening sticker 150 according to another modified form of this invention, in which (A) is a top plan view thereof and (B) is a side view thereof. The fastening sticker 150 has a printed design formed by a pattern 160 and a mark for a fastening position, i.e. a registration mark 170 which are situated on the surface of a base sheet 151 having a polygonal overall shape, and has sheet materials 152a and 152b bonded to its back surface and having male members 152a1 and 152b1, respectively. The fastening sticker 150 has the advantage of being easy to position for fastening, in addition to the advantages as described above.

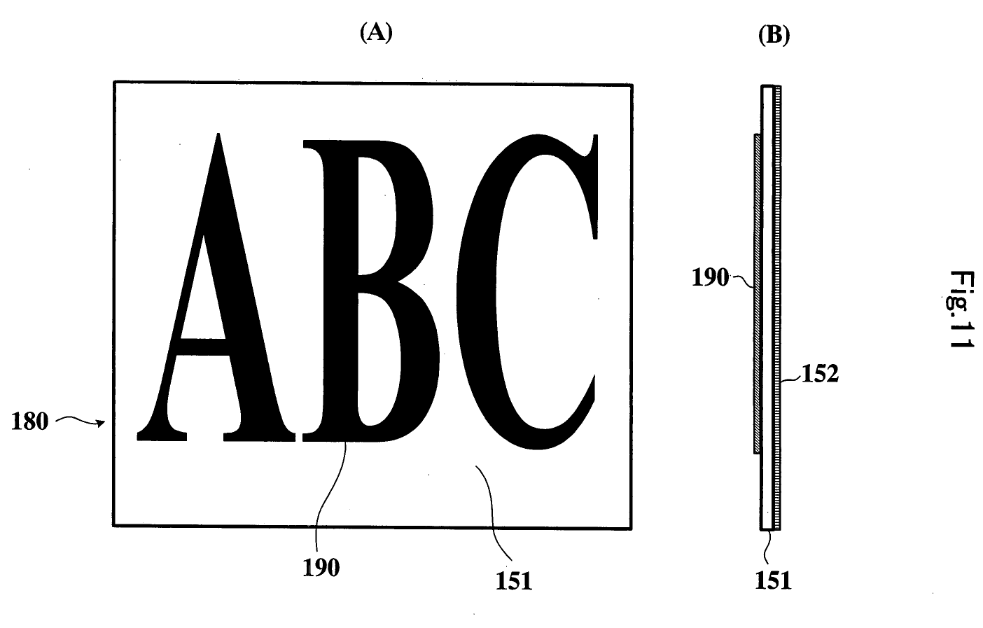

FIG. 11 shows a fastening sticker 180 according to still another modified form of this invention, in which (A) is a top plan view thereof and (B) is a side view thereof. The fastening sticker 180 has alphabetical letters 190 printed on the front surface of a base sheet 151 having a rectangular shape and has male members 152 formed on its whole back surface. According to the fastening sticker 180, since the fastening area becomes larger, the fastening power becomes greater.

Thus, the fastening sticker according to this invention may have its shape and structural features designed in various ways as required.

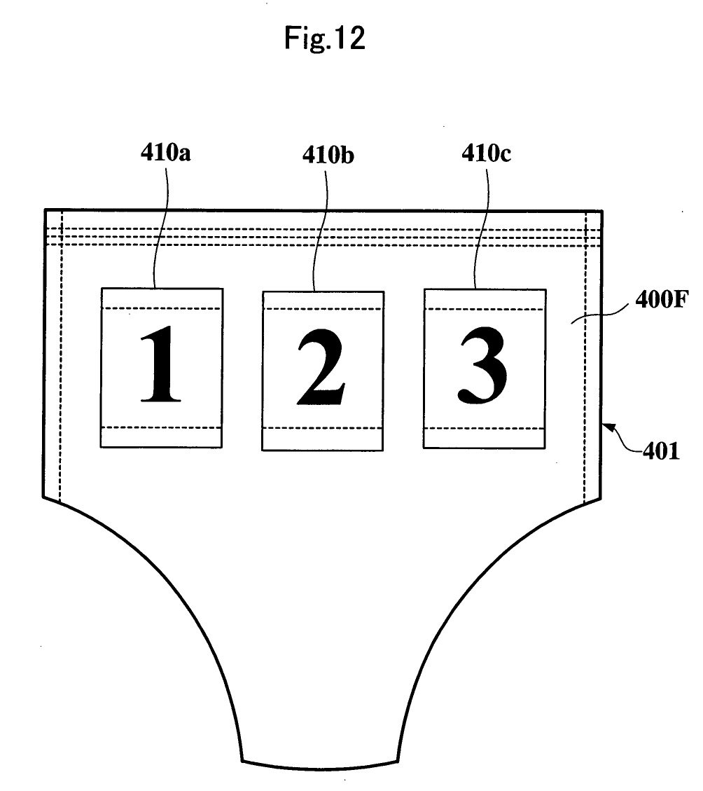

While a single fastening sticker has been shown as being fastened to each absorbent article, the fastening sticker can be increased in number as required, so that as shown by a front view of FIG. 12, for example, an absorbent article 401 may have a plurality of (in this case, three) fastening stickers 410a, 410b and 410c fastened to its front body side 400F. The use of the fastening stickers 410a, 410b and 410c prepared in a large number enables the absorbent article 201 to be folded in a still more compact shape.

The folding of the absorbent article in a compact shape is not necessarily limited to the occasion of its disposal, but it is needless to say that the fastening sticker can also be used for putting the absorbent article in order prior to use.

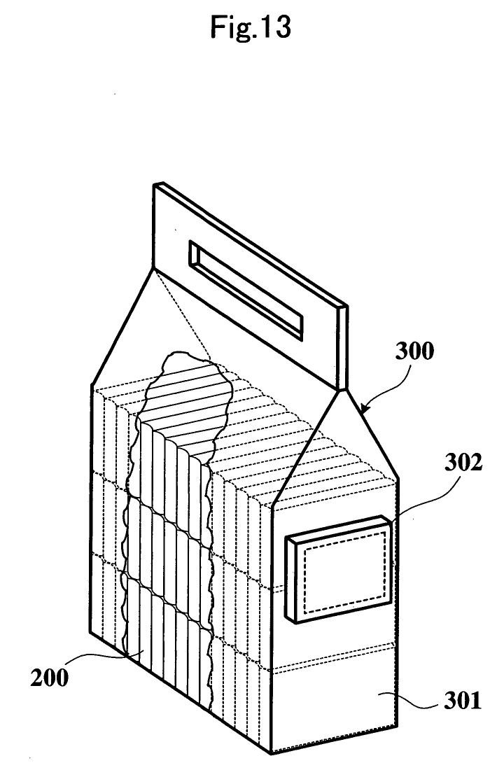

According to this invention, it is also possible to enclose various kinds of fastening stickers as described in a pocket 302 formed on the surface of a product package 300 holding a plurality of paper diaper products 200 and to be shipped to the market, for example, on one side 301 thereof, as shown by a perspective view of FIG. 13, or attach them to the product package 300 when delivering them to the user.

### INDUSTRIAL APPLICABILITY

The absorbent article according to this invention is not limited to a disposable paper diaper as described above, but is also applicable to a sanitary napkin or any of various kinds of pads, as described hereinbefore.

1. An absorbent article having a front body side and a back body side formed by folding at the longitudinal middle point of the main body of the article having an absorbent inserted between a liquid-permeable top sheet on the body side and a back sheet on the outside, the absorbent article having a fastening sticker to be used for its disposal, etc., characterized in that the fastening sticker is formed from a disposable sheet attached to the outer surface of the article, such as a sheet-like member capable of being re-stuck to and re-peeled off from the outer surface of the back sheet and separable completely from the main body of the article.

2. An absorbent article as set forth in claim 1, characterized in that the fastening sticker is fastened to the front or back body side.

3. An absorbent article as set forth in claim 1, characterized in that the fastening sticker has a design on its surface.

4. An absorbent article as set forth in claim 3, characterized in that the design is aiming the intellectual education of an infant.

5. An absorbent article as set forth in claim 3, characterized in that the design of the fastening sticker is relating to the pattern of a design on the outer surface of the main body of the article.

6. An absorbent article as set forth in claim 3, characterized in that a part of the fastening sticker on which the design is drawn or any other portion thereof is separable from the base portion of the sticker along perforations.

7. An absorbent article as set forth in claim 1, characterized in that the fastening sticker has a stretchable sheet material in its middle portion and that the sheet-like member capable of being re-stuck and re-peeled off and separable completely from the main body of the article is connected to opposite end portions of the stretchable sheet material.

8. An absorbent article as set forth in claim 1, characterized by having a plurality of such fastening stickers.

9. An absorbent article as set forth in claim 1, characterized in that the fastening sticker is attached to or enclosed in a product package for the absorbent article.
